(19) United States
(12) Patent Application Publication
(10) Pub. No.: US 2019/0290371 A1
(43) Pub. Date: Sep. 26, 2019

(54) OPTICAL SYSTEMS FOR SURGICAL PROBES, SYSTEMS AND METHODS INCORPORATING THE SAME, AND METHODS FOR PERFORMING SURGICAL PROCEDURES

(71) Applicant: MEDROBOTICS CORPORATION, Raynham, MA (US)

(72) Inventors: Thomas CALEF, Bridgewater, MA (US); Eric DALEY, Franklin, MA (US); Stephen TULLY, Milton, MA (US); Euan MORRISON, Cambridge (GB); J. Christopher FLAHERTY, Auburndale, FL (US); R. Maxwell FLAHERTY, Auburndale, FL (US)

(21) Appl. No.: 16/336,275

(22) PCT Filed: Sep. 29, 2017
(86) PCT No.: PCT/US2017/054297

§ 371 Date: May 7, 2019

Related U.S. Application Data
(60) Provisional application No. 62/401,390, filed on Sep. 29, 2016.
(60) Provisional application No. 62/481,309, filed on Apr. 4, 2017.
(60) Provisional application No. 62/504,175, filed on May 10, 2017.
(60) Provisional application No. 62/517,433, filed on Jun. 9, 2017.
(60) Provisional application No. 62/533,644, filed on Jul. 17, 2017.

(51) Int. Cl. A61B 34/30; A61B 34/00; A61B 90/00; H04N 13/133; H04N 13/239; H04N 13/25; H04N 5/225; H04N 5/232; H04N 5/235

(52) U.S. Cl. CPC A61B 34/30; A61B 34/25; A61B 90/37; H04N 13/133; H04N 13/286; H04N 13/25; H04N 5/2258; H04N 5/23296; H04N 5/2355; H04N 13/239

(57) ABSTRACT

A tool positioning system for performing a medical procedure on a patient includes an articulating probe having a distal portion and a stereoscopic imaging assembly for providing an image of a target location. The stereoscopic imaging assembly comprises: a first camera assembly comprising a first lens and a first sensor, wherein the first camera assembly is constructed and arranged to provide a first magnification of the target location; and a second camera assembly comprising a second lens and a second sensor, wherein the second camera assembly is constructed and arranged to provide a second magnification of the target location. In some embodiments, the second magnification is greater than the first magnification.

### RELATED APPLICATIONS

This application claims the benefit of U.S. Provisional Application No. 62/401,390, filed Sep. 29, 2016, the content of which is incorporated herein by reference in its entirety.

This application claims the benefit of U.S. Provisional Application No. 62/504,175, filed May 10, 2017, the content of which is incorporated herein by reference in its entirety.

This application claims the benefit of U.S. Provisional Application No. 62/517,433, filed Jun. 9, 2017, the content of which is incorporated herein by reference in its entirety.

This application claims the benefit of U.S. Provisional Application No. 62/481,309, filed Apr. 4, 2017, the content of which is incorporated herein by reference in its entirety.

This application claims the benefit of U.S. Provisional Application No. 62/533,644, filed Jul. 17, 2017, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, the content of which is incorporated herein by reference in its entirety.

This application is related to U.S. patent application Ser. No. 14/892,750, filed Nov. 20, 2015, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, the content of which is incorporated herein by reference in its entirety.

This application is related to U.S. patent application Ser. No. 13/880,525, filed Apr. 19, 2013, now U.S. Pat. No. 8,992,421, the content of which is incorporated herein by reference in its entirety.

This application is related to U.S. patent application Ser. No. 14/587,166, filed Dec. 31, 2014, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US12/40414, filed Jun. 1, 2012, the content of which is incorporated herein by reference in its entirety.

This application is related to U.S. patent application Ser. No. 14/119,316, filed Nov. 21, 2013, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, the content of which is incorporated herein by reference in its entirety.

This application is related to U.S. patent application Ser. No. 13/884,407, filed May 9, 2013, the content of which is incorporated herein by reference in its entirety.

This application is related to U.S. patent application Ser. No. 15/587,832, filed May 5, 2017, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US12/32279, filed Apr. 5, 2012, the content of which is incorporated herein by reference in its entirety.

This application is related to U.S. patent application Ser. No. 14/008,775, filed Sep. 30, 2013, the content of which is incorporated herein by reference in its entirety.

This application is related to U.S. patent application Ser. No. 14/944,665, filed Nov. 18, 2015, the content of which is incorporated herein by reference in its entirety.

This application is related to U.S. patent application Ser. No. 14/945,685, filed Nov. 19, 2015, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US12/54802, filed Sep. 12, 2012, the content of which is incorporated herein by reference in its entirety.

This application is related to U.S. patent application Ser. No. 14/343,915, filed Mar. 10, 2014, now U.S. Pat. No. 9,757,856, issued Sep. 12, 2017, the content of which is incorporated herein by reference in its entirety.

This application is related to U.S. patent application Ser. No. 15/064,043, filed Mar. 8, 2016, now U.S. Pat. No. 9,572,628, issued Feb. 21, 2017, the content of which is incorporated herein by reference in its entirety.

This application is related to U.S. patent application Ser. No. 15/684,268, filed Aug. 23, 2017, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, the content of which is incorporated herein by reference in its entirety.

This application is related to U.S. patent application Ser. No. 13/812,324, filed Jan. 25, 2013, the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US12/70924, filed Dec. 20, 2012, the content of which is incorporated herein by reference in its entirety.

This application is related to U.S. patent application Ser. No. 14/364,195, filed Jun. 10, 2014, now U.S. Pat. No. 9,364,955 issued Jun. 14, 2016, the content of which is incorporated herein by reference in its entirety.

This application is related to U.S. patent application Ser. No. 15/180,503, filed Jun. 13, 2016, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US13/54326, filed Aug. 9, 2013, the content of which is incorporated herein by reference in its entirety.

This application is related to U.S. patent application Ser. No. 14/418,993, filed Feb. 2, 2015, now U.S. Pat. No. 9,675,380 issued Jun. 13, 2017, the content of which is incorporated herein by reference in its entirety.

This application is related to U.S. patent application Ser. No. 15/619,875, filed Jun. 12, 2017, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US14/10808, filed Jan. 9, 2014, the content of which is incorporated herein by reference in its entirety.

This application is related to U.S. patent application Ser. No. 14/759,020, filed Jan. 9, 2014, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US13/43858, filed Jun. 3, 2013, the content of which is incorporated herein by reference in its entirety.

This application is related to U.S. patent application Ser. No. 14/402,224, filed Nov. 19, 2014, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US13/38701, filed May 20, 2014, the content of which is incorporated herein by reference in its entirety.

This application is related to U.S. patent application Ser. No. 14/888,541, filed Nov. 2, 2015, now U.S. Pat. No. 9,517,059, issued Dec. 13, 2016, the content of which is incorporated herein by reference in its entirety.

This application is related to U.S. patent application Ser. No. 15/350,549, filed Nov. 14, 2016, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US14/36571, filed May 2, 2014, the content of which is incorporated herein by reference in its entirety.

This application is related to U.S. patent application Ser. No. 14/888,189, filed Oct. 30, 2015, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US14/67091, filed Nov. 24, 2014, the content of which is incorporated herein by reference in its entirety.

This application is related to U.S. patent application Ser. No. 15/038,531, filed May 23, 2016, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US15/34424, filed Jun. 5, 2015, the content of which is incorporated herein by reference in its entirety.

This application is related to U.S. patent application Ser. No. 15/315,868, filed Dec. 2, 2016, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US16/28374, filed Apr. 20, 2016, the content of which is incorporated herein by reference in its entirety.

This application is related to U.S. patent application Ser. No. 11/630,279, filed Dec. 20, 2006, published as U.S. Patent Application Publication No. 2009/0171151, the content of which is incorporated herein by reference in its entirety.

### BACKGROUND

As less invasive medical techniques and procedures become more widespread, medical professionals such as surgeons may require articulating surgical tools, such as endoscopes, to perform such less invasive medical techniques and procedures that access interior regions of the body via a body orifice such as the mouth.

### SUMMARY

In an aspect, a tool positioning system for performing a medical procedure on a patient includes an articulating probe and a stereoscopic imaging assembly for providing an image of a target location. The stereoscopic imaging assembly comprises: a first camera assembly comprising a first lens and a first sensor, wherein the first camera assembly is constructed and arranged to provide a first magnification of the target location; and a second camera assembly comprising a second lens and a second sensor, wherein the second camera assembly is constructed and arranged to provide a second magnification of the target location. In some embodiments, the second magnification is greater than the first magnification.

In some embodiments, the articulating probe comprises an inner probe comprising multiple articulating inner links and an outer probe surrounding the inner probe and comprising multiple articulating outer links.

In some embodiments, one of the inner probe or the outer probe is configured to transition between a rigid mode and a flexible mode, and the other of the inner probe or the outer probe is configured to transition between a rigid mode and a flexible mode and to be steered.

In some embodiments, the outer probe is configured to be steered.

In some embodiments, the tool positioning system further comprises a feeder assembly to apply forces to the inner and outer probes.

In some embodiments, the forces cause the inner and outer probes to independently advance or retract.

In some embodiments, the forces cause the inner and outer probes to independently transition between the rigid mode and the flexible mode.

In some embodiments, the forces cause the other of the inner or outer probes to be steered.

In some embodiments, the feeder assembly is positioned on a feeder cart.

In some embodiments, the tool positioning system further comprises a user interface.

In some embodiments, the user interface is configured to transmit commands to the feeder assembly to apply the forces to the inner and outer probes.

In some embodiments, the user interface comprises a component selected from the group consisting of: joystick; keyboard; mouse; switch; monitor, touchscreen; touch pad; trackball; display; touchscreen; audio element; speaker; buzzer; light; LED; and combinations thereof.

In some embodiments, the tool positioning system further comprises a working channel positioned between the multiple inner links and the multiple outer links and wherein the stereoscopic imaging assembly further comprises a cable positioned in the working channel. In some embodiments, at least one of the outer links comprises a side lobe positioned at an outer portion thereof, the side lobe including a side lobe channel, wherein the stereoscopic imaging assembly further comprises a cable positioned in the side lobe channel.

In some embodiments, the articulating probe is constructed and arranged to be inserted into a natural orifice of the patient.

In some embodiments, the articulating probe is constructed and arranged to be inserted through an incision in the patient.

In some embodiments, the articulating probe is constructed and arranged to provide subxiphoid entry into the patient.

In some embodiments, the tool positioning system further comprises an image processing assembly configured to receive a first image captured by the first camera assembly at the first magnification and a second image captured by the second camera assembly at the second magnification.

In some embodiments, the image processing assembly is configured to generate a two-dimensional image from the first image and the second image, the two-dimensional image having a magnification that is variable between the first magnification and the second magnification.

In some embodiments, the two-dimensional image is generated by merging at least a portion of the first image with at least a portion of the second image.

In some embodiments, as the magnification of the two-dimensional image increases from the first magnification to the second magnification, a greater percentage of the two-dimensional image is formed from the second image.

In some embodiments, at the first magnification, approximately fifty percent of the two-dimensional image is formed from the first image and approximately fifty percent of the two-dimensional image is forming from the second image.

In some embodiments, at the second magnification, approximately zero percent of the two-dimensional image is formed from the first image and approximately 100 percent of the two-dimensional image is formed from the second image.

In some embodiments, at a magnification between the first magnification and the second magnification, a lower percentage of the two-dimensional image is formed from the first image than from the second image.

In some embodiments, the magnification of the two-dimensional image is continuously variable between the first magnification and the second magnification.

In some embodiments, the first sensor and the second sensor are selected from the group consisting of charge-coupled devices (CCD), complementary metal oxide semiconductor (CMOS) devices and fiber optic-bundled sensor devices.

In some embodiments, the first camera assembly and the second camera assembly are mounted within a housing.

In some embodiments, the tool positioning system further comprises at least one LED mounted in the housing.

In some embodiments, the tool positioning system further comprises a plurality of LEDs mounted in the housing, each capable of providing differing levels of light to the target location.

In some embodiments, each of the plurality of LEDs is configured to be adjustable to provide greater light output to darker areas detected in the target image and lesser light output to lighter areas detected in the target location.

In some embodiments, the stereoscopic imaging assembly is rotatably mounted within a housing at the distal portion of the articulating probe, the housing further comprising a biasing mechanism mounted between the housing and the stereoscopic imaging assembly for applying a biasing force to the stereoscopic imaging assembly and an actuation mechanism mounted between the housing and the stereoscopic imaging assembly for rotating the stereoscopic imaging assembly within the housing in conjunction with the biasing force.

In some embodiments, the biasing mechanism comprises a spring.

In some embodiments, the actuation mechanism comprises a linear actuator.

In some embodiments, the tool positioning system further comprises an image processing assembly comprising an algorithm configured to digitally enhance the image.

In some embodiments, the algorithm is configured to adjust an image parameter selected from the group consisting of: size; color; contrast; hue; sharpness; pixel size; and combinations thereof.

In some embodiments, the stereoscopic imaging assembly is configured to provide a 3D image of the target location.

In some embodiments, a first image of the target location is captured by the first camera assembly and a second image of the target location is captured by the second camera assembly; the system being configured to manipulate a characteristic of the first image to substantially correspond to a characteristic of the second image and to combine the manipulated first image with the second image to generate a three-dimensional image of the target location.

In some embodiments, a first image of the target location is captured by the first camera assembly having a first field of view and a second image of the target location is captured by the second camera assembly having a second field of view, the second field of view being narrower than the first field of view; the system being configured to manipulate the first field of view of the first image to substantially correspond to the second field of view of the second image and to combine the manipulated first image with the second image to generate a three-dimensional image of the target location.

In some embodiments, the stereoscopic imaging assembly comprises a functional element.

In some embodiments, the functional element comprises a transducer.

In some embodiments, the transducer comprises a component selected from the group consisting of: solenoid; heat delivery transducer; heat extraction transducer; vibrational element; and combinations thereof.

In some embodiments, the functional element comprises a sensor.

In some embodiments, the sensor comprises a component selected from the group consisting of: temperature sensor; pressure sensor; voltage sensor; current sensor; electromagnetic field sensor; optical sensor; and combinations thereof.

In some embodiments, the sensor is configured to detect an undesired state of the stereoscopic imaging assembly.

In some embodiments, the tool positioning system further comprises: a third lens, constructed and arranged to provide a third magnification of the target location; and a fourth lens constructed and arranged to provide a fourth magnification of the target location; wherein a relationship between the third and fourth magnifications are different than a relationship between the first and second magnifications.

In some embodiments, the first and second sensors are in fixed positions within the stereoscopic imaging assembly and the first, second, third and fourth lenses are mounted within a rotatable bezel within the stereoscopic imaging assembly; and in a first configuration, the first and second lenses are positioned to direct light to the first and second sensors and, in a second configuration, the third and fourth lenses are positioned to direct light to the first and second sensors.

In some embodiments, the first camera assembly comprises a first value for a camera parameter, and the second camera assembly comprises a second value for the camera parameter, and wherein the camera parameter is selected from the group consisting of: field of view; f-stop; depth of focus; and combinations thereof.

In some embodiments, the first value compared to the second value is relatively equal to a magnification ratio of the first camera assembly to the second camera assembly.

In some embodiments, the first lens of the first camera assembly and the second lens of the second camera assembly are each positioned in the distal portion of the articulating probe.

In some embodiments, the first sensor of the first camera assembly and the second sensor of the second camera assembly are both positioned in the distal portion of the articulating probe.

In some embodiments, the first sensor of the first camera assembly and the second sensor of the second camera assembly are both positioned proximal to the articulating probe.

In some embodiments, the tool positioning system further comprises an optical conduit optically connecting the first lens to the first sensor and the second lens to the second sensor.

In some embodiments, the second magnification is an integer value greater than the first magnification.

In some embodiments, the second magnification is twice the first magnification.

In some embodiments, the first magnification is 5× and the second magnification is 10×.

In some embodiments, the first magnification is less than 7.5× and the second magnification is at least 7.5×.

In some embodiments, the target location comprises a location selected from the group consisting of: esophageal tissue; vocal chords; colon tissue; vaginal tissue; uterine tissue; nasal tissue; spinal tissue such as tissue on the anterior side of the spine; cardiac tissue such as tissue on the posterior side of the heart; tissue to be removed from a body; tissue to be treated within a body; cancerous tissue; nasal tissue; tissue and combinations thereof.

In some embodiments, the tool positioning system further comprises an image processing assembly.

In some embodiments, the image processing assembly further comprises a display.

In some embodiments, the image processing assembly further comprises an algorithm.

In some embodiments, the tool positioning system further comprises an error detection process for notifying a user of the system of one or more failures in the operation of the first and second camera assemblies during a procedure.

In some embodiments, the error detection process is configured to monitor operation of the first and second camera assemblies and, upon detecting a failure of one of the first and second camera assemblies, enabling the user to continue the procedure using the other of the first and second camera assemblies.

In some embodiments, the error detection process is further configured to monitor operation of the other of the first and second camera assemblies and to cease the procedure upon detecting a failure of the other of the first and second camera assemblies.

In some embodiments, the error detection process comprises an override function.

In some embodiments, the tool positioning system further comprises a diagnostic function for determining a calibration diagnostic of the first and second camera assemblies.

In some embodiments, the diagnostic function is configured to: receive a first diagnostic image of a calibration target from the first camera assembly and a second diagnostic image of the calibration target from the second camera assembly; process the first and second diagnostic images to identify corresponding features; perform a comparison of the first and second diagnostic images based on the corresponding features; and if the first and second diagnostic images differ by more than a predetermined amount, determining that the calibration diagnostic has failed.

In some embodiments, the tool positioning system further comprises a depth map generation assembly.

In some embodiments, the depth map generation assembly is configured to: receive a first depth map image of the target location from the first camera assembly and a second depth map image of the target location from the second camera assembly, the first and second camera assemblies being a known distance away from each other; and generate a depth map corresponding to the target location such that, the greater a disparity between a location in the first depth map image and a corresponding location in the second depth map image, the greater the depth associated with the location.

In some embodiments, the depth map generation assembly comprises a time of flight sensor aligned with an image sensor, the time of flight sensor configured to provide a depth of each pixel of an image corresponding to a portion of the target location to generate a depth map of the target location.

In some embodiments, the depth map generation assembly comprises a light-emitting device emitting a predetermined light pattern on the target location and an image sensor for detecting the light pattern on the target location; the depth map generation assembly configured to calculate a difference between the predetermined light pattern and the detected light pattern to generate the depth map.

In some embodiments, the system is further configured to generate a three-dimensional image of the target location using the depth map.

In some embodiments, the system is further configured to: rotate a first image captured by the first camera assembly to a desired position; rotate the depth map to align with the first image in the desired position; generate a second rotated image by applying the rotated depth map to the rotated first image; and generate a three-dimensional image from the rotated first and second rotated images.

In some embodiments, at least one of the first and second sensors is configured to capture image data at a first exposure amount in a first set of pixel lines of the at least one of the first and second sensors and image data at a second exposure amount in a second set of pixel lines of the at least one of the first and second sensors.

In some embodiments, the first set of pixel lines are odd-numbered pixel lines of the at least one of the first and second sensors and the second set of pixel lines are even-numbered pixel lines of the at least one of the first and second sensors.

In some embodiments, the first exposure amount is a high exposure amount and the second exposure amount is a low exposure amount.

In some embodiments, the first exposure amount is utilized in darker areas of an image and the second exposure amount is utilized in lighter areas of the image.

In some embodiments, the imaging assembly requires power, and the system further comprises a power source remote from the imaging assembly, wherein the power is transmitted to the image assembly via a power conduit.

In some embodiments, the tool positioning system further comprises an image processing assembly, wherein image data is recorded by the imaging assembly and transmitted to the image processing assembly via the power conduit.

In some embodiments, the tool positioning system further comprises a differential signal driver configured to AC couple the image data to the power conduit.

In another aspect, a stereoscopic imaging assembly for providing an image of a target location, comprises: a first sensor mounted within a housing; a second sensor mounted within the housing; and a variable lens assembly rotatably mounted within the housing, wherein, at various positions of the variable lens assembly, image data at different levels of magnification is provided to each of the first and second sensors by the variable lens assembly.

In some embodiments, the variable lens assembly comprises an Alvarez lens.

In another aspect, a method for capturing an image of a target location, comprises providing an articulating probe comprising a distal portion, and providing a stereoscopic imaging assembly, a portion of the which is positioned at the distal portion of the articulating probe, for providing an image of a target location. The stereoscopic imaging assembly may comprise: a first camera assembly comprising a first lens and a first sensor, wherein the first camera assembly is constructed and arranged to provide a first magnification of the target location; and a second camera assembly comprising a second lens and a second sensor, wherein the second camera assembly is constructed and arranged to provide a second magnification of the target location, wherein the second magnification is greater than the first magnification. The distal portion of the articulating probe is positioned at the target location; and the image at the target location is captured using the stereoscopic imaging assembly.

In some embodiments, the method further comprises providing the captured image at a user interface.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIGS. 1A and 1B are partial schematic, partial perspective illustrative views of an articulating probe system in accordance with an embodiment of inventive concepts;

FIG. 2 is an end view of a stereoscopic image assembly system in accordance with an embodiment of inventive concepts;

FIG. 3 is a schematic diagram of the stereoscopic image assembly in accordance with an embodiment of inventive concepts;

FIG. 4 is a flowchart illustrating a 3D image generation process in accordance with an embodiment of inventive concepts;

FIGS. 5A and 5B are schematic diagrams illustrating image data captured by different camera assemblies in accordance with an embodiment of inventive concepts;

FIG. 5C is a schematic diagram illustrating a concept of combining image data to create a magnified image in accordance with an embodiment of inventive concepts;

FIG. 5D is a graph illustrating the influence of each camera assembly on a resulting 3D image in accordance with an embodiment of inventive concepts;

FIG. 6 is a flowchart illustrating a redundancy feature in accordance with an embodiment of inventive concepts;

FIG. 7 is a flowchart illustrating a diagnostic procedure in accordance with an embodiment of inventive concepts;

FIG. 8 is an end view diagram of another embodiment of the stereoscopic image assembly having a rotating lens housing in accordance with an embodiment of inventive concepts;

FIG. 9 is an end view diagram of another embodiment of the stereoscopic image assembly having a rotating lens housing in accordance with an embodiment of inventive concepts;

FIGS. 10A-10C are end view diagrams of another embodiment of the stereoscopic image assembly having a horizon correction feature in accordance with an embodiment of inventive concepts;

FIG. 11 is a schematic diagram of an image sensor in accordance with an embodiment of inventive concepts;

FIG. 12 is a flowchart illustrating a high dynamic range feature in accordance with an embodiment of inventive concepts;

FIGS. 13A-13E are schematic diagrams illustrating a concept of rotating image axes;

FIGS. 14A-14D are perspective diagrams illustrating a concept of creating a depth map from multiple images of a target area in accordance with embodiments of inventive concepts;

FIGS. 14E-14F are illustrations of a generated depth map and an associated native image from a camera assembly in accordance with embodiments of inventive concepts;

FIG. 15 is a flowchart illustrating a process for depth mapping of 2D images in accordance with an embodiment of inventive concepts;

FIG. 16 is a perspective illustrative view of an articulating probe system, in accordance with embodiments of inventive concepts;

FIGS. 17A-17C are graphic demonstrations of an articulated probe device, in accordance with embodiments of inventive concepts;

FIG. 18 is a perspective view of a line of sight robotic surgical device, in accordance with embodiments of inventive concepts;

FIG. 19 is a perspective view of an endoscopic device, in accordance with embodiments of inventive concepts; and

FIG. 20 is a schematic diagram of a portion of the stereoscopic image assembly in accordance with an embodiment of inventive concepts.

### DETAILED DESCRIPTION OF EMBODIMENTS

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

It will be further understood that when a first element is referred to as being “in”, “on”, “at” and/or “within” a second element, the first element can be positioned: within an internal space of the second element, within a portion of the second element (e.g. within a wall of the second element); positioned on an external and/or internal surface of the second element; and combinations of one or more of these, but is not limited thereto.

To the extent that functional features, operations, and/or steps are described herein, or otherwise understood to be included within various embodiments of the inventive concept, such functional features, operations, and/or steps can be embodied in functional blocks, units, modules, operations and/or methods. And to the extent that such functional blocks, units, modules, operations and/or methods include computer program code, such computer program code can be stored in a computer readable medium, e.g., such as non-transitory memory and media, that is executable by at least one computer processor.

In the following description, references are made to the capturing, manipulation and processing of images. It will be understood that this may refer to single, still images and may also refer to an image as a single frame in a video stream. In the latter case, the video stream may be comprised of many images as frames in the stream.

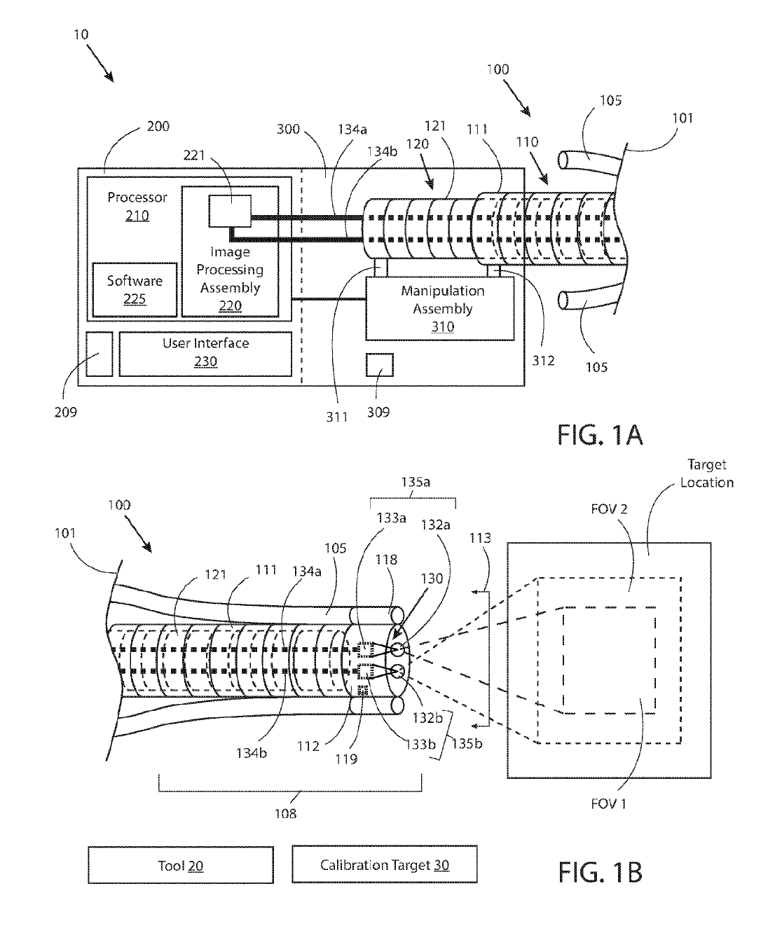

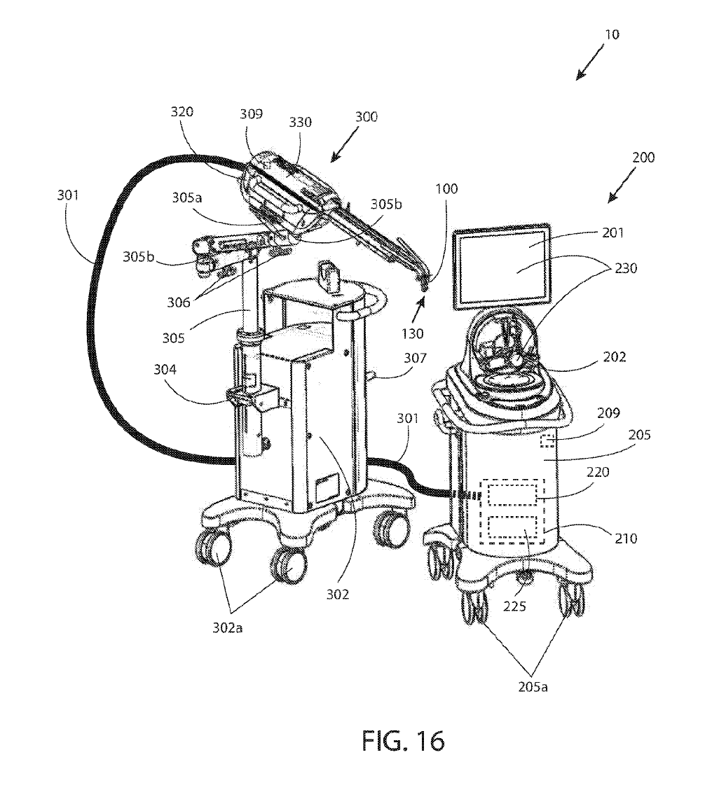

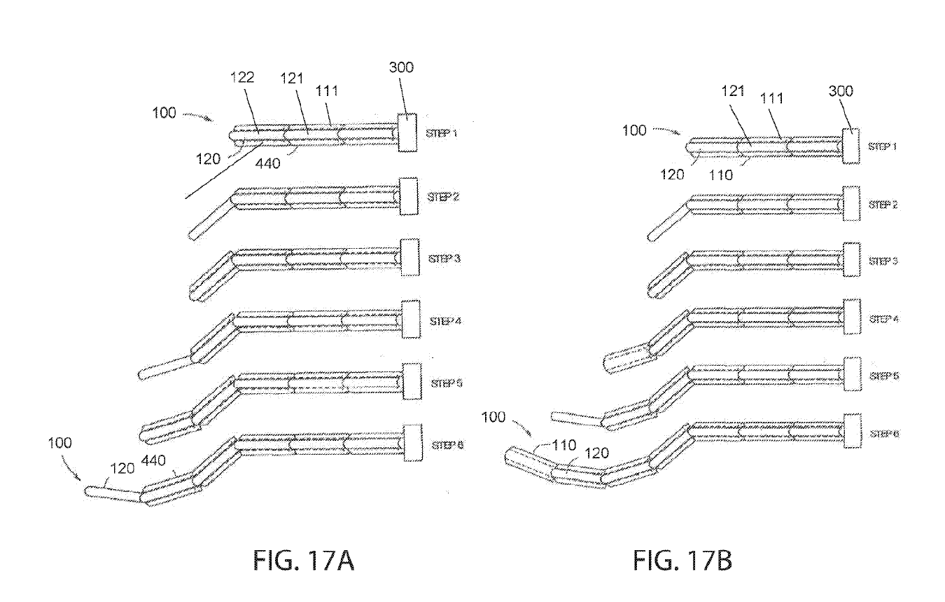

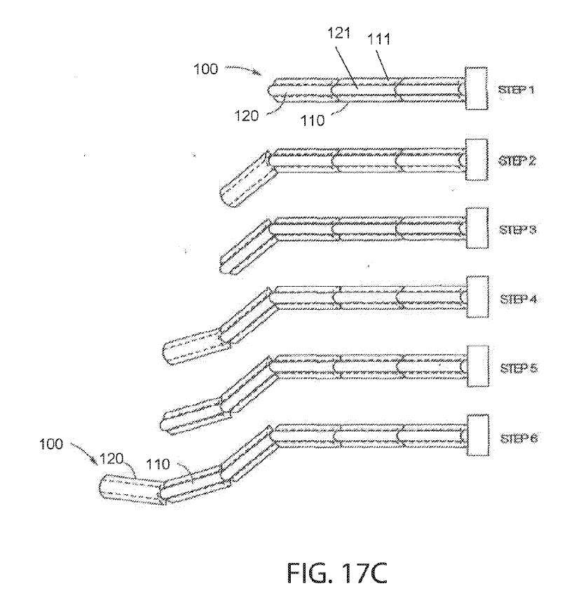

FIGS. 1A and 1B are partial schematic, partial perspective illustrative views of articulating probe system 10 according to an embodiment of inventive concepts. FIGS. 1A and 1B, when connected at line 101, illustrate an embodiment of the articulating probe system 10. As described above, in some embodiments, the articulating probe system 10 comprises a feeder unit 300 and an interface unit 200. As shown in FIGS. 1A and 1B, feeder unit 300 may include articulating probe 100, including outer probe 110, including outer links 111 and inner probe 120, including inner links 121. A manipulation assembly 310 may include a plurality of driving motors and cables positioned in the feeder unit 300, which enable the operator of the articulating probe 100 to maneuver the probe in the manner discussed above with reference to FIGS. 16 and 17A-17C. Specifically, inner control connector 311 may include cables and wiring for enabling the operator to control the movement of the inner probe 120 and outer control connector 312 may include cables and wiring for enabling the operator to control the movement of the outer probe 110, based on inputs to the manipulation assembly 310.

Interface unit 200 may include a processor 210, including software 225. Software 225 can include one or more algorithms, routines, and/or other processes (“algorithms” herein), for execution by processor 210, which enable the operation of the articulating probe system 10 described herein. User interface 230 of interface unit 200 may correspond to human interface device HID 202 for receiving tactile commands from a surgeon, technician and/or other operator of system 10, and display 201 for providing visual and/or auditory feedback, as shown in FIG. 16. Interface unit 200 may further include an image processing assembly 220, including an optical receiver 221, for receiving and processing optical signals. Optical signals are input to the optical receiver 221 over optical conduits 134a and 134b, which receive image information from camera assemblies 135a and 135b, respectively. Camera assemblies 135a and 135b are described in detail below. Optical conduits 134a and 134b may include any type of conduit capable of transmitting optical information from the camera assemblies 135a and 135b to optical receiver 221 for processing in image processing assembly 220. Power may also be supplied to the camera assemblies 135a, 135b over the conduits 134a, 134b. Examples of such conduits may include optical fiber and other data transmitting cables. Interface unit 200 and feeder unit 300 may further include functional elements 209 and 309, respectively, for providing additional inputs to the articulating probe system 10 to further enhance the manipulation and positioning of the articulating probe 100. Examples of such functional elements may include, but not be limited to, accelerometers and gyroscopes.

FIG. 1B is a perspective view of a distal portion 108 of articulating probe 100. Shown in FIG. 1B are outer links 111 of outer probe 110 and inner links 121 (shown as dashed lines) of inner probe 120. Guide tubes 105 extend along distal portion 108 and terminate at side ports 118. Guide tubes 105 and side ports 118 enable an operator of the articulating probe system 10 to introduce and position tools 20 at the end of the articulating probe 100 to perform various procedures.

When performing investigative or surgical procedures, it is imperative that the operator of the articulating probe 100 have a clear and, at certain points in a procedure, magnified view of the environment through which the articulating probe is guided and of the inspection or surgical site itself during a procedure. Typical environments also referred to as “target locations” include anatomical locations with tissue types selected from the group consisting of: esophageal tissue; vocal chords; colon tissue; vaginal tissue; uterine tissue; nasal tissue; spinal tissue such as tissue on the anterior side of the spine; cardiac tissue such as tissue on the posterior side of the heart; tissue to be removed from a body; tissue to be treated within a body; cancerous tissue; nasal tissue; tissue; and combinations thereof. It is important that the operator be able to zoom in on, or magnify, the site to ensure precision and to facilitate better intra-operative decisions. A challenge comes from the difficulty to provide a true optical zoom, which provides higher magnification, while also providing the same or better optical detail to the user. Movable zoom lenses, which include multiple lenses that are moved relative to one another to change the magnification of the system, are commonly used to enable a user of a camera system to zoom in on or magnify an object. However, such lens systems, even in miniaturized form, may be too bulky to be used in procedures such as the type of procedures that the articulated probe 100 is used to perform. Such systems may also be very expensive and, in a case where the feeder top assembly 330 (of FIG. 16) or the articulated probe 100 is intended to be disposable after being used in a procedure, it is important to manage and minimize the costs involved in the use of the articulating probe system 10. Additionally, such systems may not be capable of providing a three-dimensional image to the operator. Another option might be to provide a digital zoom through software manipulation. However, digitally zooming involves an interpolation algorithm, which blurs the image and may reduce the optical clarity of the image.

Distal portion 108 of articulating probe 100 may include a stereoscopic imaging assembly 130 coupled to distal outer link 112, including a first camera assembly 135a and a second camera assembly 135b. According to aspects of the inventive concept, camera assemblies 135a, 135b may each include a fixed-magnification lens 132a, 132b and an optical assembly 133a, 133b. Optical assemblies 133a, 133b may be charge-coupled devices (CCD), complementary metal oxide semiconductor (CMOS) devices, fiber optic-bundled systems, or any other technology suitable for this application.

According to an embodiment of the inventive concept, lenses 132a and 132b may have different levels of magnification. For example, lens 132a may have a first magnification that provides a first field of view, FOV1, and lens 132b may have a second magnification that provides a second field of view, FOV2. As shown in FIG. 1B, in an embodiment, field of view FOV1 of lens 132a is narrower than field of view FOV2 of lens 132b. This may be a result of lens 132a having a greater magnification than lens 132b. For example, lens 132b may have a magnification of 5X and lens 132a may have a magnification of 10X. It will be understood, however, that any combination of magnifications of the lenses may be used, as long as the lenses have different magnification levels. It is important to note that the camera assemblies 135a, 135b may be aligned and oriented with respect to each other to be centered and focused on the same point of a target location. As is described in greater detail below, the use of multiple camera assemblies having different magnification levels enables the image processing assembly 220 to manipulate the image data received from each camera assembly to produce images magnified at the magnification level of each of the lenses 132a, 132b, as well as at magnification levels there between. The use of multiple camera assemblies also enables the image processing assembly 220 to manipulate the image data received from each camera assembly to produce three-dimensional images of the target location viewed by the stereoscopic image assembly 130. In some embodiments, first camera assembly 135a comprises a first value for a camera parameter, and second camera assembly 135b comprises a second value for the (same) camera parameter. In these embodiments, the camera parameter can be a parameter selected from the group consisting of: field of view; f-stop; depth of focus; and combinations thereof. The ratio of the two values can be relatively equal to the magnification ratio of the two camera assemblies.

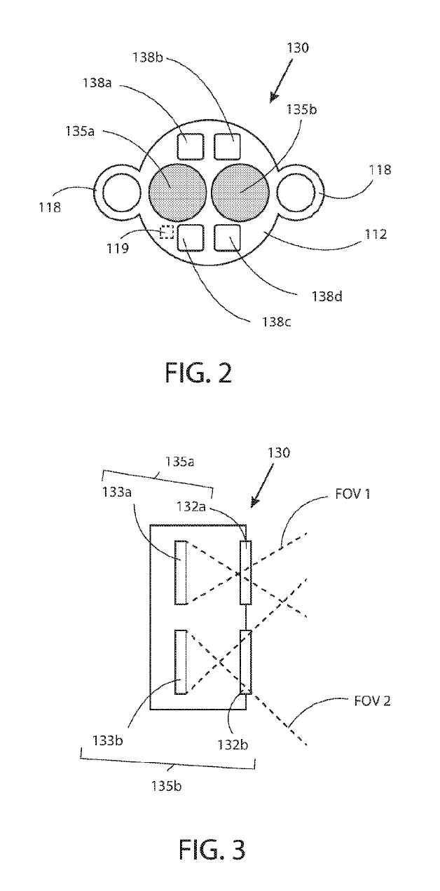

FIG. 2 is an end view of the stereoscopic image assembly 130 as seen from line 113 of FIG. 1B. Shown are side ports 118, as well as stereoscopic image assembly 130, which includes camera assemblies 135a and 135b. Stereoscopic image assembly 130 may also include a number of LEDs 138a-d for providing illumination, for the camera assemblies 135a, 135b, of the path of travel of the articulating probe 100, as well as the target location, once the articulating probe 100 is situated in the location of the procedure to be performed. While four LEDs 138a-138d are shown in FIG. 2, it will be understood that fewer LEDs or more LEDs may be used in the stereoscopic image assembly 130. Further, more than two camera assemblies may be incorporated into the stereographic image assembly 130, each having a different magnification level, but all focused on a similar point of the target location. A functional element 119 may also be included, for providing additional inputs to the articulating probe system 10 to further enhance the manipulation and positioning of the articulating probe 100. Examples of such functional elements may include, but not be limited to, accelerometers and gyroscopes.

According to an aspect of the inventive concept, LEDs 138a-138d may be controlled individually to optimize the view provided to the operator and to the stereographic image assembly 130. Upon receiving images from the optical assemblies 133a, 133b, the processor 210, based on an image analysis performed by the image processing assembly 220, may vary the intensity of light provided by each LED 138, to enable uniform exposure across the image. In another embodiment, pixel illumination in each quadrant of the optical assembly may be analyzed and the output of corresponding LEDs controlled to optimize the resulting images.

FIG. 3 is a schematic diagram of the stereoscopic image assembly 130, including camera assemblies 135a and 135b. As shown, camera assembly 135a may include lens 132a and optical assembly 133a. Based on the magnification level of lens 132a, camera assembly 135a has the field of view FOV1. Likewise, camera assembly 135b may include lens 132b and optical assembly 133b. Based on the magnification level of lens 132b, camera assembly 135b has the field of view FOV2. In an embodiment, when the magnification of lens 132a is twice the magnification of lens 132b, field of view FOV1 is a factor of, for example half of, field of view FOV2. Different ratios of magnification between the lenses will yield different, proportional differences in the fields of view. For example, camera assembly 135a may have a 40-degree field of view and provide 10× of magnification, while camera assembly 135b may have an 80-degree field of view and provide 5× of magnification.

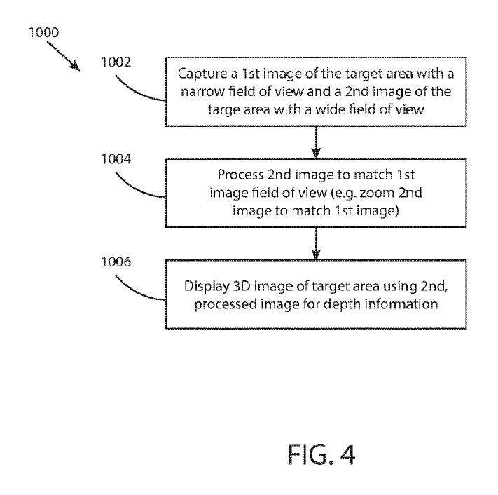

The two-dimensional images captured by each of the camera assemblies 135a and 135b are transmitted to image processing assembly 220 via optical conduits 134a and 134b, respectively, and optical receiver 221. According to an aspect of the inventive concept, the received 2D image frames may be processed by the image processing assembly 220 to produce corresponding 3D image frames. This process is generally shown in flowchart 1000 of FIG. 4. In Step 1002, a first image of a target area is captured by camera assembly 135a, which, as described above, has a narrow field of view FOV1. A concurrent, corresponding second image of the target area is captured with camera assembly 135b, which has a wider of view FOV2. In Step 1004, the second image may be processed so that it matches the field of view of the first image. This processing may involve digitally magnifying, or increasing the zoom of, the second image, so that it matches the field of view FOV1 of the first image. A 3D image may be generated, in a conventional manner, in Step 1006, using a combination of the first, narrow field of view image and the digitally-zoomed second image. The digitally-zoomed second image is used to provide depth information to the viewer of the combined 3D image. While some resolution is lost in the second image when it is digitally zoomed, it is known in the field of 3D imaging that the viewer can effectively perceive a 3D image while viewing images of varying resolution. A higher resolution image (the narrow field of view image as described) provides clarity to the viewer, while the lower resolution image provides depth cues. Therefore, for the purposes contemplated in various embodiments, the articulating probe system 10 is effectively able to provide a lossless 3D video image at the magnification level of the narrow field of view camera.

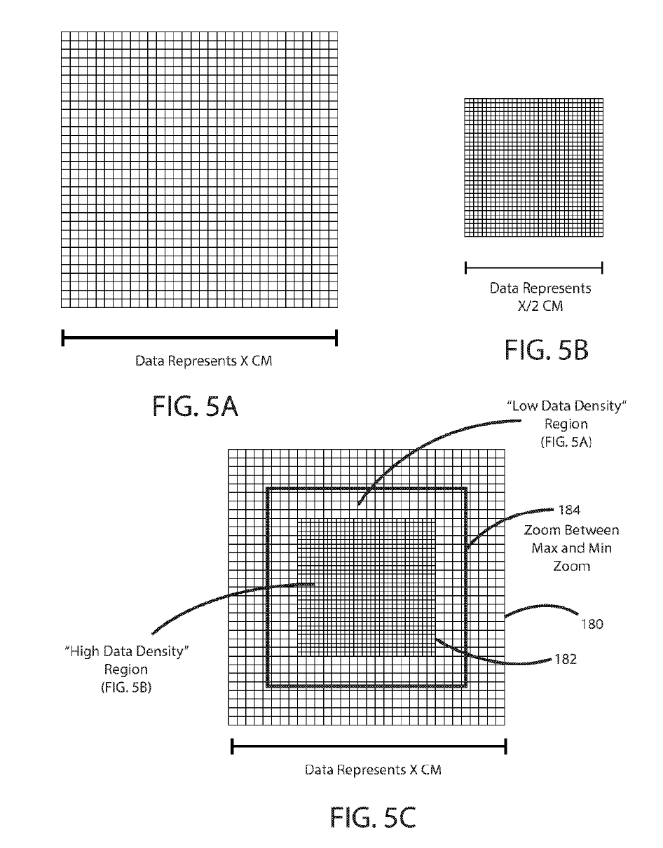

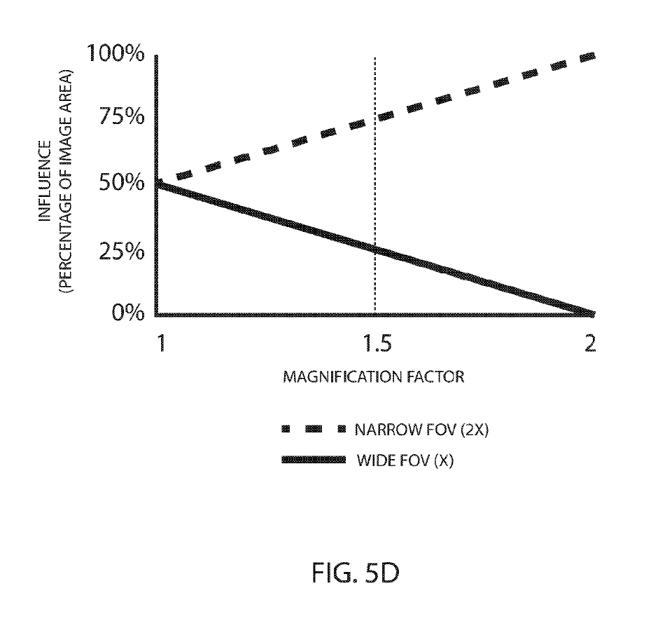

The multiple-camera system also enables the generation of an image capable of having a range of simulated continuous magnification levels between the magnification level of each camera assembly 135a, 135b, by combining image data from each of the camera assemblies. The configuration of images of various magnification levels is described with reference to FIGS. 5A-D. Shown in FIG. 5A is a graphical representation of image data captured by camera assembly 135b, having the wide FOV (FOV2) lens and FIG. 5B is a graphical representation of image data captured by camera assembly 135a, having the narrow FOV (FOV1) lens. As shown in FIG. 5A, the representation of image data includes a larger area, however, as the number of pixels are held constant, the resolution of the image captured will be lower, as shown by the size of the grid within the image square. As shown in FIG. 5B, when using the narrow FOV (FOV1) lens of assembly 135a, the area of captured image data is smaller and evenly distributed over the same number of pixels as previously mentioned. This results in an image having less area, but higher resolution higher resolution than the image captured by the wide FOV (FOV2) lens of assembly 135b. Continuing with the example above, the wide FOV2 image data shown in FIG. 5A is twice the area of the narrow FOV1 image data shown in FIG. 5B.

Typically, the user performing a surgical procedure is concerned mostly with the middle of the visible workspace displayed on display 201. Inserting the higher resolution image of FIG. 5B in the middle of the lower resolution image of FIG. 5A provides a better visualization of the area of interest. To ensure that the user still has the ability to see and work with a larger area, the low data density region is aligned to the high data density region and displayed as the “periphery”. An example of such a configuration is shown in FIG. 5C

With the two images overlaid, as shown in FIG. 5C, the center of the final “image” may have a higher data density (dots per inch, or representative pixels per inch), and the outer portion, sourced from the camera assembly 135b with the lower zoom level, or wide FOV2, may have a lower data density (fewer dots per inch, or fewer representative pixels per inch). In order to simulate a “zoomed” or magnified image of a size similar to the size of the image generated by camera assembly 135b, as shown in FIG. 5A, a portion of this “image” would then be chosen (based on the desired zoom level) to be displayed to the user, and as the graphics card displayed the image, areas of lower data density (the periphery image data) would be less crisp than the areas of higher data density (the center image data, corresponding to the FOV1 image from camera assembly 135a).

FIG. 5D is a graph showing the amount of image source influence that each camera assembly 135a, 135b contributes to a resulting image output from the image processing assembly 220, depending on the magnification selected for the image. The dashed line depicts the percent influence of camera assembly 135a, the narrow field of view (FOV1) camera, and the solid line depicts the percent influence of camera assembly 135b, the wide field of view (FOV2) camera. At a relative magnification factor of 1, which in the example described above is 5×, 50% of the image output from the image processing assembly 220 consists of the image captured by camera assembly 135a and 50% of the image consists of the image captured by camera assembly 135b. This is shown at 180 in FIG. 5C. As can be seen in FIG. 5C, the center 50% portion of the total image 180 comprises 100% of the narrow field of view (FOV1) image from camera assembly 135a, and the outer 50% of image 180 comprises 100% of the wide field of view (FOV2) image from camera assembly 135b. However, since the image data from camera assembly 135a covers or replaces the center 50% of the image data from camera assembly 135b, only 50% of the FOV2 image is displayed and visible to the user. Accordingly, in the resulting image 180, the center 50% of the image comprises the FOV1 image from camera assembly 135a and the outer 50% of the image comprises the FOV2 image from camera assembly 135b.

Likewise, at a relative magnification factor of 2, which in the example described above is 10×, the image output from the image processing assembly 220 consists of approximately 100% of the FOV1 image captured by camera assembly 135a, with approximately 0% contribution by the FOV2 image captured by camera assembly 135b. This is shown at 182 in FIG. 5C. In this instance, the image displayed to the user may be scaled up by processing software 225 to a size accommodated by the display 201.

At magnification levels in between 5× and 10×, the images captured by camera assemblies 135a and 135b contribute to the output-magnified image based on the proportion of the magnification level. For example, for an output image at 7.5× (or a relative magnification factor of 1.5, shown at 184 in FIG. 5C and by the dotted line in FIG. 5D), the center 75% of the image output from the image processing assembly 220 comprises approximately 100% of the FOV1 image captured by camera assembly 135a, and the outer 25% of the image comprises a portion of the FOV2 image captured by camera assembly 135b. To scale to a magnification factor of 1.5 (7.5× magnification) in this example, the outer 25% of the FOV2 image is cropped to enable the FOV1 image to contribute a greater percentage to the resulting image 184. Since the image data from camera assembly 135a covers or replaces the center 75% of the image data from camera assembly 135b, only approximately 25% of the FOV2 image is displayed and visible to the user.

For an output image lower than 7.5×(or a relative magnification factor of 1.5) the FOV1 image captured by narrow field camera assembly 135a makes up a lower percentage of the resulting output image and the FOV2 image captured by wide field camera assembly 135b makes up a higher percentage of the resulting output image. Likewise, for an output image higher than 7.5× (or a relative magnification factor of 1.5) the FOV1 image captured by narrow field camera assembly 135a makes up a higher percentage of the resulting output image and the FOV2 image captured by wide field camera assembly 135b makes up a lower percentage of the resulting output image.

Generally, an image output by image processing assembly 220 may comprise approximately 100% of the FOV1 image captured by camera assembly 135a, which may make up between approximately 50% and 100% of the output image, depending on the magnification factor applied to the output image. Further, depending on the magnification factor applied to the output image, between approximately 0% and 50% of the output image may comprise at least a portion of the FOV2 image captured by camera assembly 135b. Magnifications closer to a magnification factor of 1 will comprise a greater portion of the FOV2 image, while magnifications closer to a magnification factor of 2 will comprise a smaller portion of the FOV2 image. In each instance, the resulting image may be scaled up or down in size by processing software 225 to a size accommodated by the display 201.

In an embodiment having more than two camera assemblies, more image data may be utilized to provide the generated zoom images at magnifications between those provided by each camera assembly.

To provide further granularity during the continuous zoom phase, the output image may be further improved with a number of image processing features that provide a digital enhancement of the image. Examples may include sizing, detail, color and other parameters.

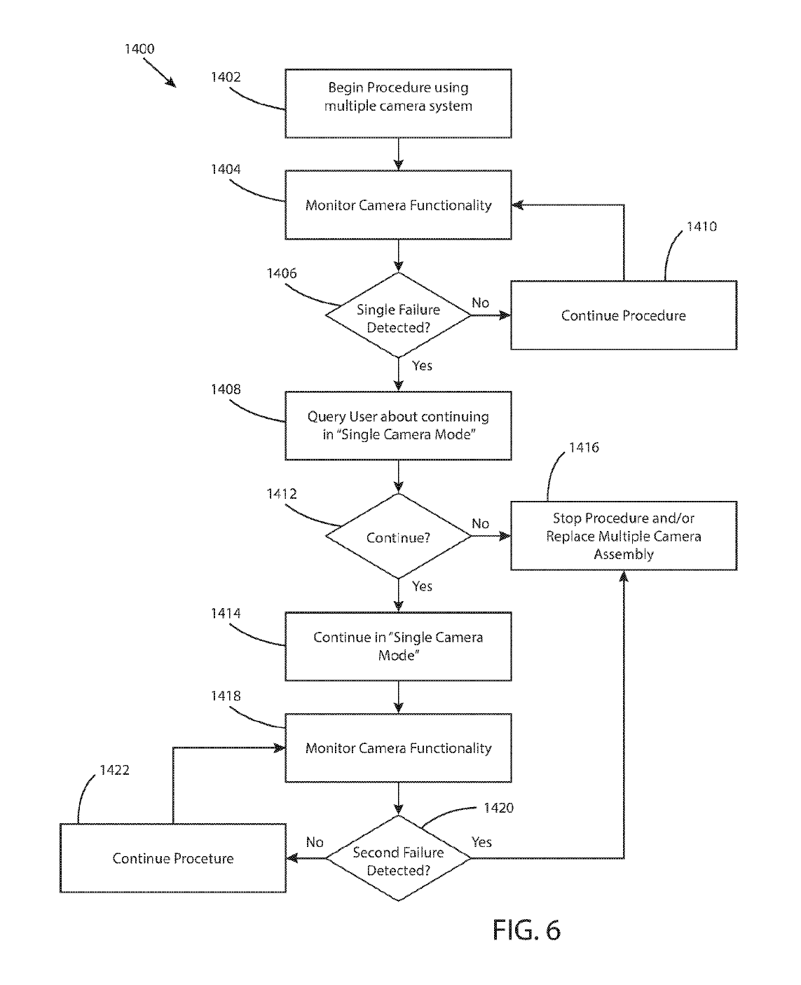

According to another aspect of the present inventive concept, the stereoscopic image assembly 130 may include an error detection process that may provide a redundancy feature for the articulating probe system 10. Accordingly, in the event that one of camera assemblies 135a, 135b fails during a procedure, the operator would be given the option to continue the procedure using the single operating camera assembly, for example by using an override function provided by the error detection process. This process is depicted in flowchart 1400 of FIG. 6. In Step 1402, a procedure may be started using the articulating probe 100 with both camera assemblies 135a, 135b operating. Processor 210 continuously monitors the functionality of both camera assemblies, Step 1404. If a failure is not detected, Step 1406, the operator is able to continue the procedure, Step 1410.

However, if, in Step 1406, a failure of one of the camera assemblies 135a, 135b is detected, the operator may be notified of the failure through the user interface 230 and queried about continuing the procedure using only the remaining operable camera assembly, Step 1408. If the operator chooses not to continue in Step 1412, the procedure is terminated for replacement of the faulty camera assembly, Step 1416. If, in Step 1412, the operator chooses to continue the procedure, which choice may be communicated to the processor 210 via the user interface 230, the procedure is continued in a “single camera mode,” Step 1414. Processor 210 continues to monitor the functionality of the remaining camera assembly, Step 1418. As long as a second failure is not detected, Step 1420, the procedure is continued, Step 1422. If a second failure is detected in Step 1420, the procedure is terminated, Step 1416. In connection with the foregoing, a failure could be any type of degradation of the ability of a camera assembly to provide optimal quality images, for example, complete mechanical or electrical failure or even the associated lens being fouled with debris that prevents it from operating properly.

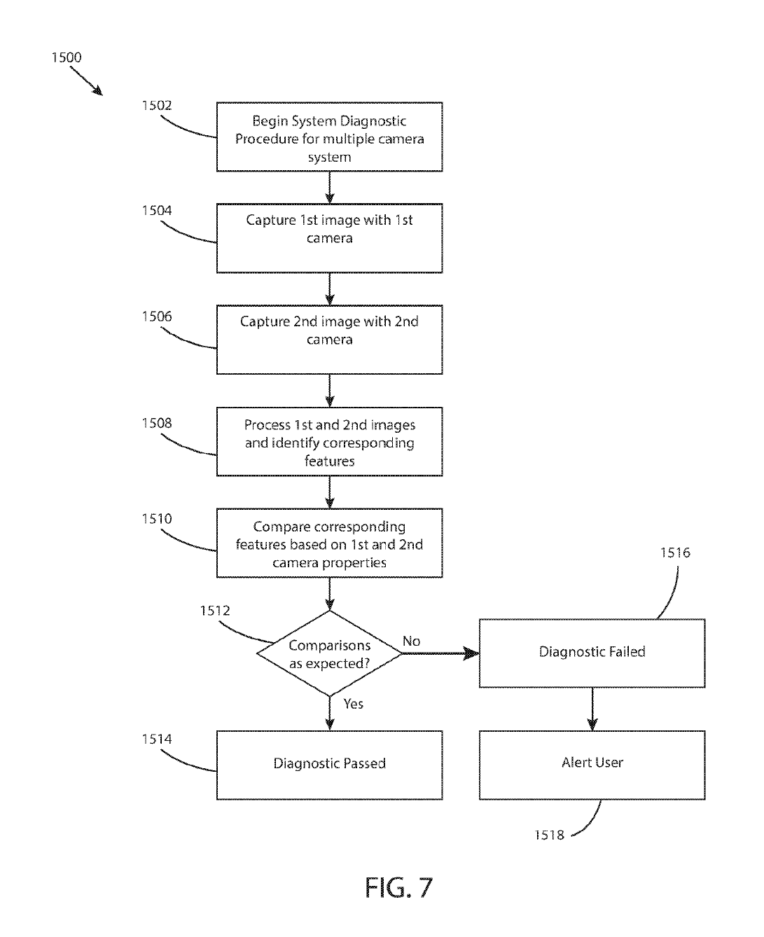

In order to ensure that both camera assemblies 135a, 135b are operating properly, a system diagnostic procedure may be undertaken. An example calibration procedure will now be described with reference to flowchart 1500 of FIG. 7. At Step 1502, the diagnostic procedure is commenced. In Step 1504, a first image of a target object may be captured using the first camera assembly 135a. The image captured may be of any target object or pattern that can be captured by both camera assemblies. The target should have sufficient detail to enable a thorough diagnostic test of the camera assemblies. In an embodiment, a calibration target 30 (FIG. 1B) may be used at the beginning of a procedure. In Step 1506, a second image of the target object may be captured with the second camera assembly 135b. The first and second images may be processed by image processing assembly 220 to identify features of the images, Step 1508, and the identified features of the first and second images are compared to each other, Step 1510. If the comparison of the identified features of the first and second images are as expected (i.e., they correspond to each other, relative to the magnification properties of each camera assembly), Step 1512, the system is deemed to have passed the diagnostic procedure, Step 1514, and the procedure is allowed to continue. If, however, the comparison reveals that features of the first and second images are not as expected, Step 1512, the system is deemed to have failed the diagnostic procedure, Step 1516, and the user or operator is alerted of the failure, Step 1518.

This procedure may be undertaken at the beginning of each procedure, and also periodically or continuously throughout the procedure. The data acquired through the diagnostic procedure may be utilized in the functionality monitoring procedure described with reference to FIG. 6.

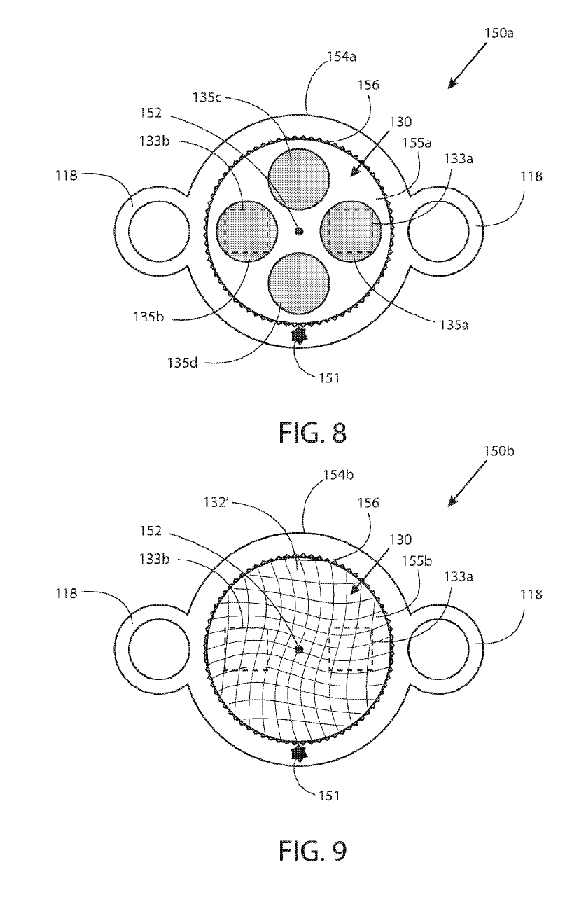

FIG. 8 is an end view of another embodiment of the stereoscopic image assembly 130 as seen from line 113 of FIG. 1B, in which multiple sets of paired lenses may be maneuvered to be used in conjunction with an associated optical assembly. Distal outer link 150a may include a stationary outer housing 154a and a rotating lens housing 155a. Stereoscopic image assembly 130 may include two optical assemblies 133a, 133b. However, rotating lens housing 155a may include four lenses 135a-135d, and each may provide a different field of view and magnification level. In an embodiment, as will become apparent lenses 135a and 135b operate as a pair and lenses 135c and 135d operate as a pair. In a first position, shown in FIG. 8, lenses 135a and 135b are positioned over optical assemblies 133a and 133b, respectively. In this orientation, image processing assembly 220 receives images from each of the optical assemblies 133a, 133b and is able to process the image data to produce images at the magnification level of lens 135a, at the magnification level of lens 135b, or any magnification level there between, using the procedure described above. In this position of rotating lens housing 155a, lenses 135c and 135d are not positioned over an optical assembly and therefore, they do not contribute to images captured by the stereoscopic image assembly 130.

Outer link 150a further may include a motor (not shown) for driving a gear 151, which is mated to outer teeth configuration 156 of rotating lens housing 155a. As described above, lenses 135a-135d may have different magnification levels. Therefore, to change the zoom range of images captured by the optical assemblies 133a and 133b, rotating lens housing 155a may be rotated 90 degrees about an axis 152 by driving gear 151, to position lenses 135c and 135d over optical assemblies 133b and 133a, respectively. This may provide the stereoscopic image assembly 130 with a different range of magnification than that provided by lenses 135a and 135b.

FIG. 9 is an end view diagram of another embodiment of the stereoscopic image assembly 130 as seen from line 113 of FIG. 1B. Distal outer link 150b may include a stationary outer housing 154b and a rotating lens housing 155b. Stereoscopic image assembly 130 may include two optical assemblies 133a, 133b. However, rotating lens housing 155b may include an Alvarez-type variable focus lens 132′ rather than the multiple lenses described above. Outer link 150b further may include a motor (not shown) for driving a gear 151, which is mated to outer teeth configuration 156 of rotating lens housing 155b. In order to provide different levels of magnification to each of the optical assemblies 133a and 133b, a movable portion of the lens 132′ may be rotated about axis 152 by gear 151, relative to a fixed portion of the lens 135′. The lens 132′ may be configured such that variable, known levels of magnification are provided to each of the optical assemblies 133a and 133b. The processing of images obtained with this configuration may be similar to that described above.

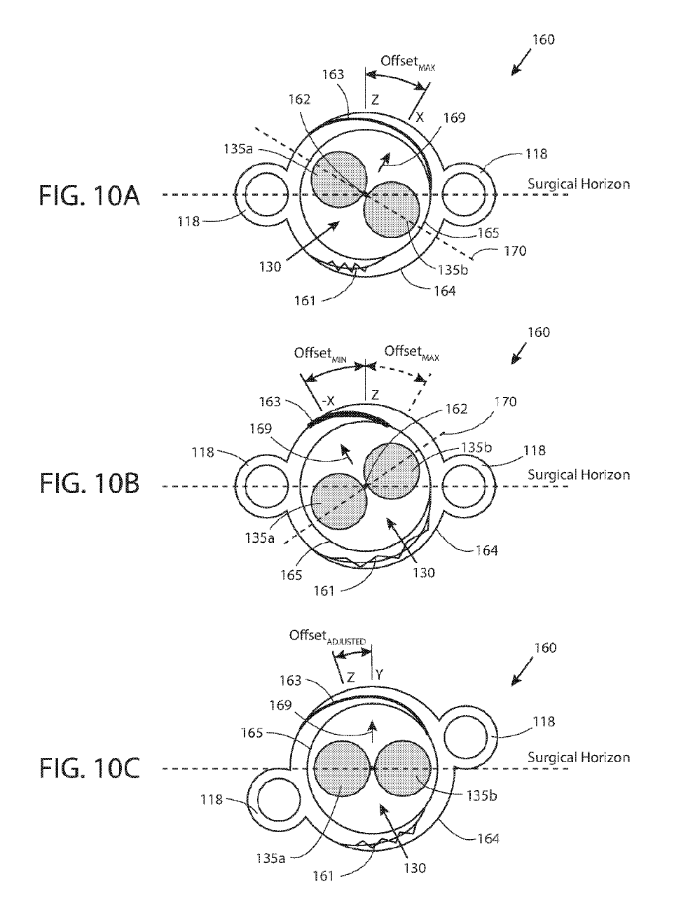

FIGS. 10A-10C are end view diagrams of another embodiment of the stereoscopic image assembly 130, as seen from line 113 of FIG. 1B, having a horizon correction feature. During a procedure in which the articulating probe 100 is being maneuvered, link-by-link, to a target location through a natural orifice or a surgeon-created orifice through tissue toward a target area, it is possible for the orientation of the distal outer link, that houses the stereographic image assembly 130, to rotate to an orientation outside of the “surgical horizon,” or the expected plane of view of the surgeon. In other words, an axis of the camera assemblies 135a and 135b may become askew relative to the expected planar positioning of the camera assemblies 135a and 135b. When this occurs, it is very difficult to turn the stereographic image assembly 130 by rotating the entire articulating probe 100, and it can also be difficult to rotate a 3D image. Therefore, it is important that the stereographic image assembly 130 be easily and quickly rotatable so that the camera axis is aligned with the surgical horizon, both for visual orientation purposes for the operator, as well as for enabling the system to acquire proper image data for generating 3D images.

As shown in FIG. 10A, the camera axis, indicated by camera axis 170, which bisects camera assemblies 135a and 135b, is not in line with the surgical horizon. However, distal outer link 160 may include a horizon correction apparatus that enables the stereographic image assembly 130 to be rotated about a central axis 162 to correct the orientation of the stereographic image assembly 130 and to line up the camera assemblies 135a, 135b with the surgical horizon.

Stereographic image assembly 130 may be rotatable within a rotatable housing 165, within housing 164 of distal link 160, about central axis 162. A biasing spring 161 may be attached at one end to housing 164 and at the other end to stereographic image assembly 130 to provide a biasing force between the two components. Countering the biasing force is a linear actuator 163, also coupled between the housing 164 and the stereographic image assembly 130. Linear actuator 163 may comprise a device having a length that is electrically or mechanically controllable to enable it to exert a force against the biasing force provided by the spring 161, which enables the stereographic image assembly 130 to be controllably rotated within the housing 164. Examples of such linear actuators may be a solenoid device, a nitinol wire, or other device having similar properties. The biasing spring 161 is configured to allow a known amount of positive and negative offset from a position of the camera in which the camera axis 170 bisecting the camera assemblies 135a and 135b, is aligned with the surgical horizon. Such a position is shown in FIG. 10C. In this position, which is also indicated when arrow 169 points straight up, the camera axis 170 is aligned with the surgical horizon.

Referring back to FIG. 10A, shown is the situation where the stereographic image assembly 130 is tilted to the maximum offset X from an aligned position Z allowed by the biasing spring 161 and linear actuator 163. As shown, biasing spring 161 may be in a semi-relaxed state, and linear actuator 163 is extended to a length that enables the maximum offset of X. To align the camera axis 170 with the surgical horizon, the length of the linear actuator 163 may be shortened and the stereographic image assembly 130 rotated, against the biasing force of the spring 161, until the camera axis 170 is aligned with the surgical horizon. FIG. 10B illustrates a situation where the stereographic image assembly 130 is tilted to the minimum offset −X from an aligned position Z allowed by the biasing spring 161 and linear actuator 163. As shown, biasing spring 161 is in an extended state, and linear actuator 163 is shortened to a length that enables the minimum offset of −X. To align the camera axis 170 with the surgical horizon, the length of the linear actuator 163 is increased and the stereographic image assembly 130 is rotated, aided by the biasing force of the spring 161, until the camera axis 170 is aligned with the surgical horizon.

FIG. 10C illustrates an intermediate position of the stereographic image assembly 130, where the length of the linear actuator 163 has been manipulated to cause the stereographic image assembly 130 to rotate an amount Y to an adjusted position, where the camera axis 170 and the surgical horizon are aligned.

During surgical procedures, the lighting requirements can change drastically and quickly. In some cases, the amount of light required to fully illuminate the surgical field may be beyond the capability of a lighting system associated with the stereographic image assembly 130. To compensate for low-light or high-light conditions, the exposure parameters of the optical assemblies 133a, 133b may be altered to allow the pixels in the sensors in the optical assemblies 133a, 133b more or less time to integrate the photons that are received into a signal that is relayed to the image processing assembly 220. For example, if the surgical site is very dark, the exposure of a sensor may be increased to allow more photons to reach the sensor and produce a brighter image. Conversely, if the surgical site is very bright, the exposure may be shortened to allow less light to reach the sensor, resulting in lower probabilities of sensor saturation.

While increasing or decreasing the exposure may account for one lighting condition at a time, in the case of positioning the articulating probe 100 and during a surgical procedure, lighting conditions can change rapidly, or within the target area within a single frame. Therefore, high dynamic range processing may be used to enable the operator to capture images with different exposures and combine them into an optimized image, compensated for the lighting variations across the optical assembly. To accomplish this, images having multiple exposure settings may be taken by alternating horizontal rows of pixels within the sensor of the optical assembly with different exposure settings.

An aspect of the inventive concept is to improve the performance of the camera assemblies 135a, 135b in high dynamic range situations while meeting the low-latency requirements for robotic surgery. This would be, for example, when certain regions of the image are very well exposed with sufficient lighting while other regions of the image are under exposed and darker. In an embodiment, a mode of each camera assembly 135a, 135b may be activated that provides alternating lines of different exposure. The odd pixel lines may be configured for a higher exposure time in order to capture greater image detail in darker regions. The even pixel lines may be configured for a lower exposure time in order to capture image detail in highly illuminated regions. It will be understood that any configuration of pixel lines and varying amounts of exposure may be utilized according to various aspects of the inventive concept. For example, every third pixel line may be configured for higher exposure time relative to a lower exposure time for two pixels lines there between. Any combination of high or low exposure times corresponding to any combination or configuration of pixel lines are considered within the scope of the inventive concept.

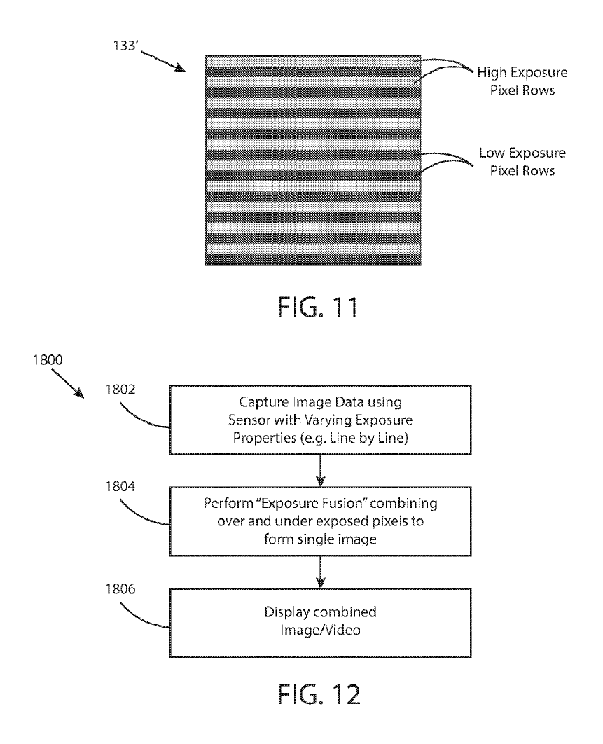

FIG. 11 is a schematic diagram of a sensor 133′ of one of the optical assemblies 133a, 133b. In an embodiment, odd numbered pixel rows are set for high exposure and even numbered pixel rows are set for low exposure. In an example, even numbered pixels rows of the sensor 133′ may have an exposure time of T, while odd numbered pixels rows may have an exposure time of 2T. As such, the odd numbered pixel rows will collect twice the light of the even numbered pixel rows. Using high dynamic range technology, the image may be manipulated by the image processing assembly to provide improved dynamic range by utilizing lighter pixels in dark areas of the image and darker pixels in lighter area of the image.

In an embodiment, the output of the camera sensor 133′ may be processed as follows: the captured image or video stream may be input to a custom image processing apparatus, for example, an FPGA designed to perform exposure fusion (the combination of the high and low exposure data) into a single processed image. Any saturated regions of the image may be better represented due to the apparatus applying a higher weighting to the short exposure data from the even pixel lines. Any dark regions may be better represented due to the apparatus applying a higher weighting to the long exposure data from the odd pixel lines. The processing may then allow for additional tone mapping of the resulting image to enhance or reduce contrast. The apparatus may use frame buffers and/or line buffers to store data for processing within the processing apparatus. The apparatus may process video in real-time with just a small additional latency due to the data buffering.

This process is outlined in flowchart 1800 of FIG. 12. In Step 1802, an image is captured using the sensor 133′ with varying exposure properties, as described above. A single image is generated, Step 1804, by combining over and under exposed pixels in an exposure fusion process. Such a process is known in the art and will not be described here. The generated image is then displayed to the operator, Step 1806.

As described above with reference to FIGS. 10A-10C, the system 10 may be able to mechanically rotate the stereographic image assembly 130 to align the camera axis 170 with the surgical horizon. In certain circumstances, it may be desirable to digitally rotate a stereoscopic image. However, given the complexities involved in generating the stereoscopic image, simply rotating each image from the camera assemblies separately may not provide a user perceivable stereoscopic image.

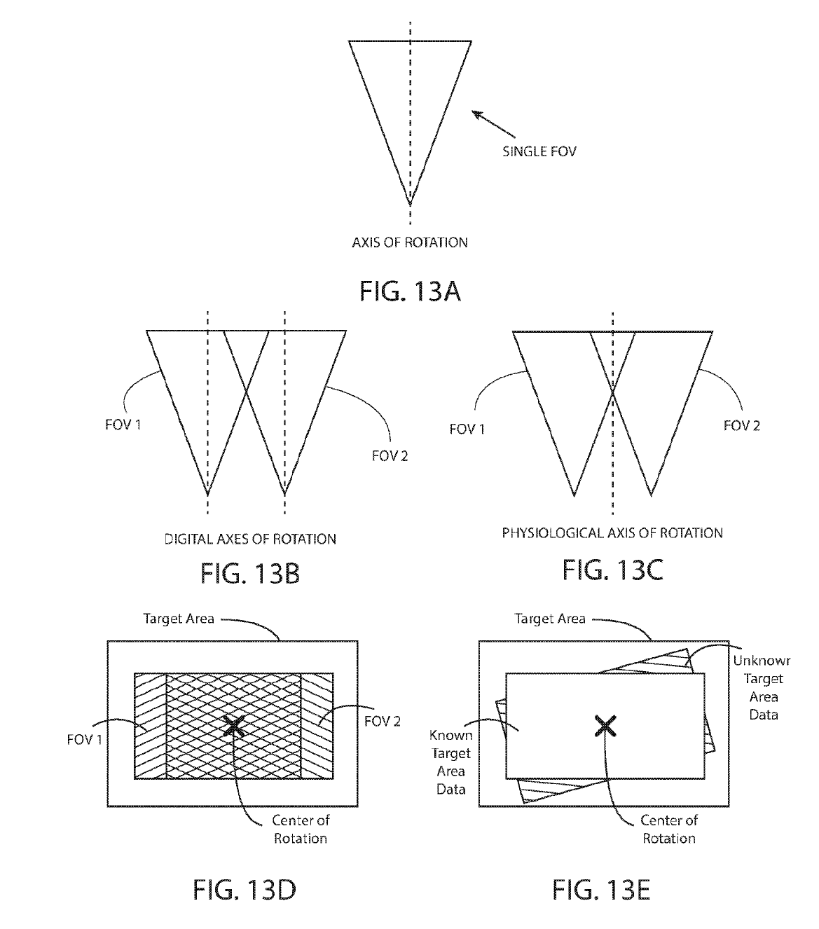

In standard 2D image rotation, the image is rotated about the center of the native image, as shown in FIG. 13A. This creates a natural and non-distracting simulation of a rotated view. 3D image rotation requires additional manipulation to create a natural simulation of a rotated view. To produce a 3D image perceivable by viewer, a stereoscopic camera system must mimic the orientation of the viewer's natural eye position and orientation (e.g. proportionally mimic the eyes). As shown in FIG. 13B, rotation about the center of each of a stereoscopic pair of images would not properly mimic the physiological rotation (e.g. tilting) of a human head and eyes, as shown in FIG. 13C, where the eyes rotate about a single, central axis. Rotating each image about its central axis would alter the relationship between the stereoscopic pair in a way that would prevent the images from “converging”, or forming an image with perceivable depth, when viewed by the user. Digitally rotating a stereoscopic pair about a shared central axis however presents separate challenges. As shown in FIGS. 13D and 13E, when rotating the stereoscopic images about the center of the pair, the “rotated” image requires information about the target area not known to the system. This information is needed to maintain images that converge as a 3D image for the user.

According to an aspect of the inventive concept, the above issues may be rectified by generating a depth map of the scene that provides a pixel-by-pixel depth representation of the captured image. In an embodiment, camera assemblies 135a, 135b each capture an image of a target area. Since the camera assemblies have a known distance between them, the view from each camera assembly, relative to a reference point, will be different from each other. A difference between the two images may be calculated relative to the reference point to generate a depth map, which in combination with one of the two images, may be used to regenerate the second of the two images (e.g. after a rotation has been performed, as described herein). The depth map along with the one image can be individually rotated, such that the regenerated image is also rotated, and the pair can be displayed as a digitally rotated stereoscopic pair.

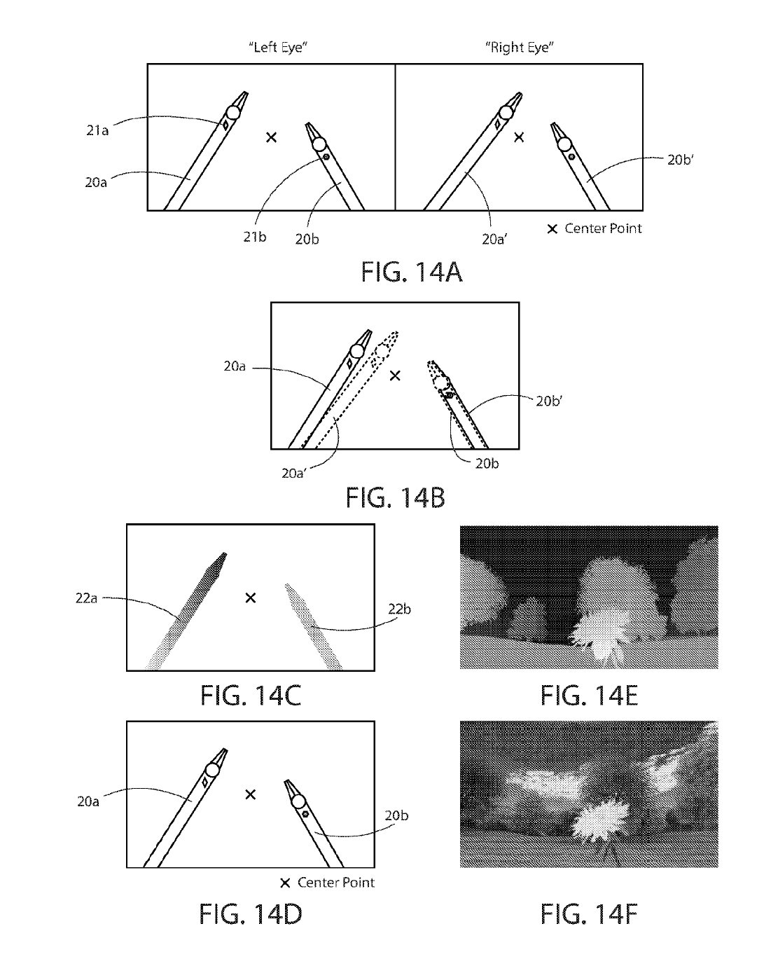

Referring now to FIGS. 14A-14F, generation of a depth map which may be used to generate separate images from camera assemblies 135a, 135b to form a rotatable stereoscopic image will be described. FIG. 14A illustrates a “left eye” image and a “right eye” image of a pair of tools 20a and 20b. The left eye image may be captured by a first camera assembly and the right eye image may be captured by a second camera assembly, where the first and second camera assemblies are in different locations and a known distance from each other (e.g. a stereoscopic pair). As can be seen in FIG. 14B, the locations of tools 20a′ and 20b′ are different in the left eye image than in the right eye image. The center point of each image at “X” is used as a reference point to determine the extent of the difference. Markings 21a and 21b may be included on tools 20a and 20b, respectively, to provide further navigational reference points used in the generation of the depth map, as described below.

FIG. 14B shows the left eye and right eye images (2D) of FIG. 14A overlain, to show the disparity of the two tools from the center as seen by each camera. As shown, tools 20a and 20b, in solid lines, represent the data from the left eye image of FIG. 14A and tools 20a′ and 20b′, in dashed lines, represent the data from the right eye image of FIG. 14A. This information may be used by image processing assembly 220 and software 225 to generate a depth map, shown in FIG. 14C. As seen, object 22a represents depth data of tool 20a of the left eye image of FIG. 14A and object 22b represents depth data of tool 20b of the left eye image of FIG. 14A.

The greater the positional disparity of the tools from the center of the left eye and right eye images (2D), the greater the depth associated with that object (or the pixels that make up that object) from the imaging system. Therefore, as shown in FIG. 14C, darker colored pixels represent portions of the image that are farther away from the stereoscopic camera pair and lighter colored pixels represent portions of the image that are closer to the stereoscopic camera pair. Accordingly, in FIG. 14C, based on the gradient from light to dark of object 22a, the system can determine that the tip of the tool 20a is farther away from the stereoscopic camera pair than the proximal end of the tool 20a. To the contrary, since the color of the pixels that make up object 22b in FIG. 14C are substantially the same, it can be determined that tool 20b is substantially parallel to the stereoscopic camera pair. FIG. 14D illustrates the left eye, which in combination with the depth map of FIG. 14C, can be processed by image processing assembly 220 to regenerate the “right eye” image of FIG. 14A.

FIGS. 14E and 14F are diagrams that further illustrate the depth map concept described above. FIG. 14E illustrates a depth map of an image captured in the same manner as that described above with reference to FIGS. 14A-14D. Software 225 examines both left and right images, determines a pixel-by-pixel depth map, by identifying like pixels in each image, determining the disparity from center, and creating the compete depth map. As shown, darker pixels represent image data more distant from the camera assemblies, while lighter pixels represent image data closer to the camera assemblies. This depth map data is combined with the image of FIG. 14F (e.g. a “left eye” image) to regenerate the “right eye” image, creating a stereoscopic pair of images to be displayed to a user to perceive as a 3D image.

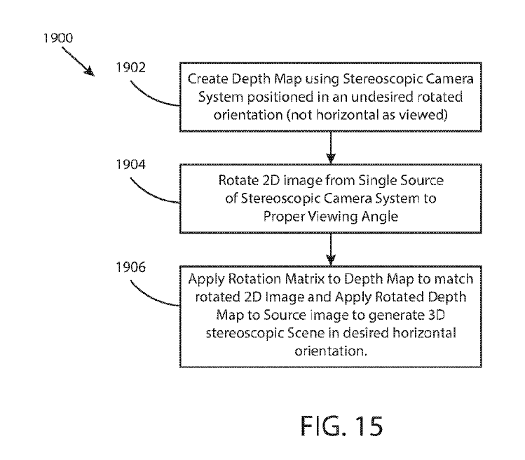

FIG. 15 is a flowchart 1900 illustrating steps involved in utilizing the depth map process described above to generate a stereoscopic image that may be digitally rotated. In Step 1902, if the stereoscopic imaging assembly 130 is positioned in an undesired rotated orientation during a procedure, (e.g. the camera axis is not aligned with the surgical horizon) a depth map of the target area may be created as described above. A first image captured by one of the camera assemblies 135a, 135b is rotated to the proper viewing angle, where the camera axis is aligned with the surgical horizon, Step 1904. A rotation matrix may then be applied to the depth map to rotate it to align with the rotated image and the depth map is applied to the first, rotated image to generate a second rotated image corresponding to the other one of the camera assemblies, resulting in a 3D stereoscopic image in the desired horizontal orientation, Step 1906.

Alternatively, a depth map may be created using an image sensor to capture a 2D image and a “time of flight” sensor that has been aligned to the image sensor. The “time of flight” sensor could provide the depth of each pixel and software could align the 2D image to the data received from the time of flight sensor to generate a depth map. Another system could include a system including a light-emitting device for emitting a light pattern that is known, and an image sensor for detecting the pattern on the target area. The system could then calculate the difference in the pattern detected by the image sensor compared to the known pattern that has been emitted, and a depth map calculated.

FIG. 16 is a perspective illustrative view of an articulating probe system 10 according to an embodiment of inventive concepts. System 10 includes articulating probe 100, comprising a stereoscopic imaging assembly 130, as described herein. In some embodiments, the articulating probe system 10 comprises a feeder unit 300 and an interface unit 200 (also referred to as console 200). The feeder unit 300, also referred to as a feeding mechanism, may be mounted to a feeder cart 302 at a feeder support arm 305. Feeder support arm 305 is adjustable in height, such as via rotation of crank handle 307 which is operably connected to vertical height adjuster 304 which slidingly connects feeder support arm 305 to feeder cart 302. Feeder support arm 305 can include one or more sub-anus or segments that pivot relative to each other at one or more mechanical joints 305b that can be locked and/or unlocked clamps 306 by one or more or related coupling devices. This configuration permits a range of angles, orientations positions, degrees of motion, and so on for positioning the feeder unit 300 relative to a patient location. In some embodiments, one or more feeder supports 305a are attached between feeder support arm 305 and feeder unit 300, such as to partially support the weight of feeder unit 300 to ease positioning feeder unit 300 relative to feeder support arm 305 (for example, when one or more joints 305b of feeder support arm 305 are in an unlocked position permitting manipulation of the feeder unit 300). Feeder support 305a may comprise a hydraulic or pneumatic support piston, similar to the gas springs used to support tail gates of automobiles or trucks. In some embodiments, two segments of feeder support arm 305 are connected with a support piston (not shown) for example a support piston positioned at one of the segments, such as to support the weight of feeder unit 300, or simply base assembly 320 alone. The feeder unit 300 may include a base assembly 320 and a feeder top assembly 330 that is removably attachable to the base assembly 320. In some embodiments, a first feeder top assembly 330 can be replaced with another or second top assembly 330, after one or more uses (e.g. in a disposable manner). A use may include a single procedure performed or a human patient or multiple procedures performed on the same patient. In some embodiments, base assembly 320 and top assembly 330 are fixedly attached to each other.

The top assembly 330 includes an articulating probe 100 for example comprising a link assembly including an inner link mechanism comprising a plurality of inner links, and an outer link mechanism comprising a plurality of outer links, as described in connection with various embodiments herein, as described herebelow in reference to FIGS. 17A-17C. In some embodiments, articulating probe 100 comprises an inner mechanism of articulating links and an outer mechanism of articulating links, such as those described in applicant's co-pending International PCT Application Serial No. PCT/US2012/70924, filed Dec. 20, 2012, or U.S. patent application Ser. No. 14/364,195, filed Jun. 10, 2014, the content of which is incorporated herein by reference in its entirety. The position, configuration and/or orientation of the probe 100 are manipulated by a plurality of driving motors and cables positioned in the base assembly 320, as described in FIG. 1 hereabove. The feeder cart 302 can be mounted on wheels 302a to allow for manual manipulation of its position. Feeder cartwheels 302a can include one or more locking features used to lock cart 302 in position after a manipulation or movement of articulating probe 100, base assembly 320, and/or other elements of feeder unit 300. In some embodiments, mounting of the feeder unit 300 to a moveable feeder cart 302 is advantageous, such as to provide a range of positioning options for an operator, versus mounting of feeder unit 300 to the operating table or other fixed structure. Feeder unit 300 can comprise a functional element 309 as described hereabove in reference to FIG. 1.

In some embodiments, the base assembly 320 is operably connected to the interface unit 200, such connection typically including electrical wires, optical fibers, or wireless communications, for transmission of power and/or data, or mechanical transmission conduits such as mechanical linkages or pneumatic/hydraulic delivery tubes, conduit 301 shown. The interface unit 200 includes a user interface 230, comprising a human interface device HID 202 for receiving tactile commands from a surgeon, technician and/or other operator of system 10, and a display 201 for providing visual and/or auditory feedback. The interface unit 200 can likewise be positioned on an interface cart 205, which is mounted on wheels 205a (e.g. lockable wheels) to allow for manual manipulation of its position. Base assembly 320 can comprise a processor, 210, including an image processing unit 220 and software 225, as described hereabove in reference to FIG. 1. Base assembly 320 can further comprise a functional element 209, also as described hereabove.

FIGS. 17A-17C are graphic demonstrations of a highly articulating probe device, according to embodiments of the present inventive concepts. A highly articulating robotic probe 100, according to the embodiment shown in FIGS. 17A-17C, comprises essentially two concentric mechanisms, an outer mechanism and an inner mechanism, each of which can be viewed as a steerable mechanism. FIGS. 17A-17C show the concept of how different embodiments of the articulating probe 100 operate. Referring to FIG. 17A, the inner mechanism can be referred to as a first mechanism or inner link mechanism 120. The outer mechanism can be referred to as a second mechanism or outer link mechanism 110. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the tetra “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume the outer link mechanism 110 is rigid and the inner link mechanism 120 is limp, as seen in step 1 in FIG. 17A. Now, the inner link mechanism 120 is both pushed forward by feeder assembly 102 (see e.g. FIG. 16), described herein, and its “head” or distal end is steered, as seen in step 2 in FIG. 17A. Now, the inner link mechanism 120 is made rigid and the outer link mechanism 440 is made limp. The outer link mechanism 110 is then pushed forward until it catches up or is coextensive with the inner link mechanism 120, as seen in step 3 in FIG. 17A. Now, the outer link mechanism 110 is made rigid, the inner link mechanism 120 limp, and the procedure then repeats. One variation of this approach is to have the outer link mechanism 110 be steerable as well. The operation of such a device is illustrated in FIG. 17B. In FIG. 17B it is seen that each mechanism is capable of catching up to the other and then advancing one link beyond. According to one embodiment, the outer link mechanism 110 is steerable and the inner link mechanism 120 is not. The operation of such a device is shown in FIG. 17C.

In medical applications, operation, procedures, and so on, once the probe 100 arrives at a desired location, the operator, such as a surgeon, can slide one or more tools through one or more working channels of outer link mechanism 110, inner link mechanism 120, or one or more working channels formed between outer link mechanism 110 and inner link mechanism 120, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of articulating probe 100, such as working channels comprising one or more radial projections extending from outer link mechanism 110, these projections including one or more holes sized to slidingly receive one or more tools. As described with reference to other embodiments, working channels may be of outer location of the articulating probe 100.

In addition to clinical procedures such as surgery, articulating probe 100 can be used in numerous applications including but not limited to: engine inspection, repair or retrofitting; tank inspection and repair; surveillance applications; bomb disarming; inspection or repair in tightly confined spaces such as submarine compartments or nuclear weapons; structural inspections such as building inspections; hazardous waste remediation; biological sample and toxin recovery; and combination of these. Clearly, the device of the present disclosure has a wide variety of applications and should not be taken as being limited to any particular application.

Inner link mechanism 120 and/or outer link mechanism 110 are steerable and inner link mechanism 120 and outer link mechanism 110 can each be made both rigid and limp, allowing articulating probe 100 to drive anywhere in three-dimensions while being self-supporting. Articulating probe 100 can “remember” each of its previous configurations and for this reason, articulating probe 100 can retract from and/or retrace to anywhere in a three dimensional volume such as the intracavity spaces in the body of a patient such as a human patient.

The inner link mechanism 120 and outer link mechanism 110 each include a series of links, i.e. inner links 121 and outer links 111 respectively, that articulate relative to each other. In some embodiments, the outer links are used to steer and lock the probe, while the inner links are used to lock the articulating probe 100. In “follow the leader” fashion, while the inner links 121 are locked, the outer links 111 are advanced beyond a distal-most inner link 122. The outer links 111 are steered into position by the system steering cables, and then locked by locking the steering cables. The cable of the inner links 121 is then released and the inner links 121 are advanced to follow the outer links. The procedure progresses in this manner until a desired position and orientation are achieved. The combined inner links 121 and outer links 111 may include working channels for temporary or permanent insertion of tools at the surgery site. In some embodiments, the tools can advance with the links during positioning of the probe. In some embodiments, the tools can be inserted through the links following positioning of the probe.

One or more outer links 111 can be advanced beyond the distal-most inner link prior to the initiation of an operator controlled steering maneuver, such that the quantity extending beyond the distal-most inner link will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of probe 100. In some embodiments, an operator can select the number of links used for steering (e.g. to select between 1 and 10 links to be advanced prior to each steering maneuver).

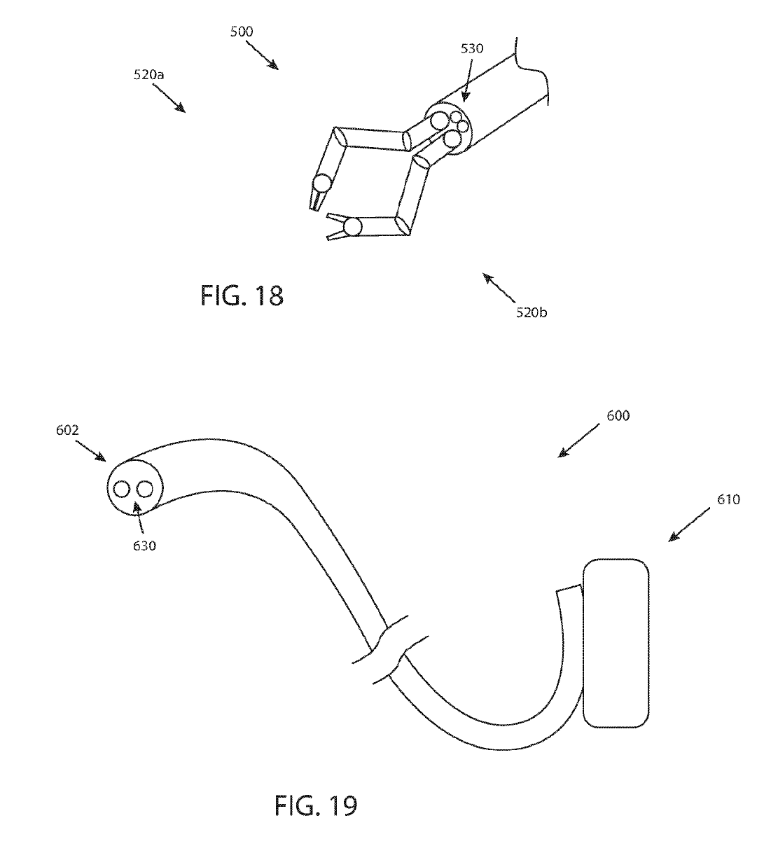

While the inventive concept has been described for use in connection with a surgical probe device, it will be understood that it is equally suitable for use in connection with any type of device where stereoscopic imaging may be advantageous or desired, such as a line-of-sight robot 500, including tools 520a, 520b and camera assembly 530, as shown in FIG. 18, and an endoscope 600, having a scope 602 including a camera assembly 630, as shown in FIG. 19.

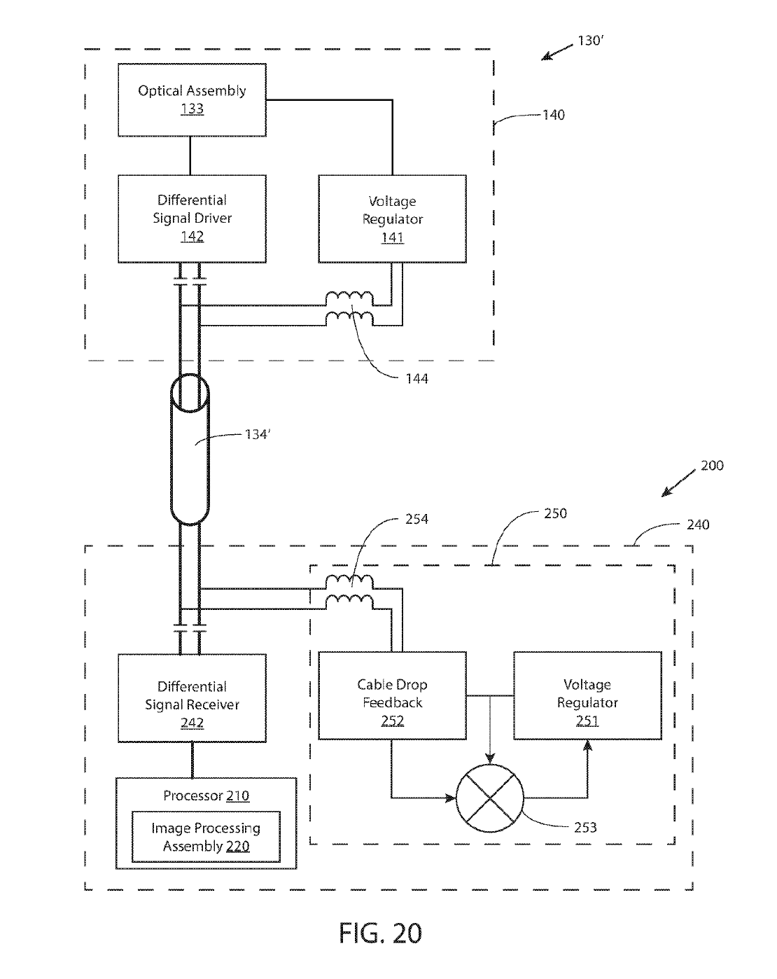

FIG. 20 is a schematic diagram of an imaging assembly and an interface unit in accordance with an embodiment of inventive concepts. As described herein, an imaging assembly 130′ may comprise one or more optical assemblies 133, (e.g. a stereoscopic imaging assembly comprises two optical assemblies). In some embodiments, each optical assembly 133 may comprise one or more electronic components, such as CCD or CMOS components. In these embodiments, imaging assembly 130′ may comprise a circuit 140, requiring a power source to enable its functionality. Power may be provided via an onboard battery, and/or via a power-carrying wire connected to an external power source, such as a power source integral to a console or base assembly as described herein. In the embodiment shown in FIG. 20, power may be provided from interface unit 200 via optical conduit 134′ comprising one or more wire pairs, such as one or more twisted pairs. Digital optical data may be transferred between imaging assembly 130′ and interface unit 200 via the same optical conduit 134′ (i.e. the same two wires transmit both power and data). Interface unit 200 comprises a circuit 240, comprising a power transmit assembly 250. Power transmit assembly 250 may include a voltage regulator 251, feedback circuit 252, combiner 253, and inductor 254, configured to provide a power source to circuit 140 via conduit 134′. Inductor 254 may be selected to limit 300-400 MHz signal noise on conduit 134′.

Circuit 140 comprises a voltage regulator 141 and inductor 144. Voltage regulator 141 is configured to receive power from transmit assembly 250 and provide power to circuit 140. Voltage regulator 141 may comprise a low-dropout (LDO) voltage regulator configured to step down the voltage provided to circuit 140. Regulator 141 is configured to provide clean, stable voltage rails for optical assembly 133. Inductor 144 may be selected to limit 300-400 MHz signal noise on conduit 134′. Circuit 140 further comprises a differential signal driver 142 that receives optical data from optical assembly 133. Differential signal driver 142 transmits the received optical data to differential signal receiver 242 by AC coupling the data to conduit 134′. Differential signal receiver 242 may decouple the optical data from conduit 134′, and transmit the data to image processing assembly 220 of processor 210.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

1. A tool positioning system, comprising:
an articulating probe;
a stereoscopic imaging assembly for providing an image of a target location, comprising:
a first camera assembly comprising a first lens and a first sensor, wherein the first camera assembly is constructed and arranged to provide a first magnification of the target location; and
a second camera assembly comprising a second lens and a second sensor, wherein the second camera assembly is constructed and arranged to provide a second magnification of the target location;
wherein the second magnification is greater than the first magnification.

2-83. (canceled)
